(19) United States
(12) Patent Application Publication
(10) Pub. No.: US 2013/0096319 A1
(43) Pub. Date: Apr. 18, 2013

(54) PROCESS FOR PREPARATION OF AMISULPRIDE

(71) Applicants: Dinesh Jayntibhai Paghdar, Pune (IN); Mahesh Ramkumar Kolekar, Pune (IN); Tushar Nandkumar Deshpande, Pune (IN); Suryaprakash Pandurang Patil, Pune (IN); Yuvraj Atmaram Chavan, Pune (IN); Purna Chandra Ray, Pune (IN); Girij Pal Singh, Pune (IN)

(72) Inventors: Dinesh Jayntibhai Paghdar, Pune (IN); Mahesh Ramkumar Kolekar, Pune (IN); Tushar Nandkumar Deshpande, Pune (IN); Suryaprakash Pandurang Patil, Pune (IN); Yuvraj Atmaram Chavan, Pune (IN); Purna Chandra Ray, Pune (IN); Girij Pal Singh, Pune (IN)

(73) Assignee: Lupin Limited, Mumbai, Maharashtra (IN)

(21) Appl. No.: 13/704,935

(22) PCT Filed: Jun. 9, 2011
(86) PCT No.: PCT/IB11/01301

§ 371 Date: Dec. 18, 2012

(30) Foreign Application Priority Data
Jun. 17, 2010 (IN) 648/KOL/2010
Dec. 1, 2010 (IN) 1365/KOL/2010

(51) Int. Cl. C07D 207/09; C07C 217/84; C07C 315/00

(52) U.S. Cl. CPC C07D 207/09; C07C 315/00; C07C 217/84
USPC 548/567; 562/430; 560/12; 560/43

(57) ABSTRACT

The present invention is related to a novel process for the preparation of amisulpride (I) which involves: methylation of 4-amino-salicylic-acid (VI) with dimethyl sulphate and base, optionally in presence of TBAB to obtain 4-amino-2-methoxy methyl benzoate (VII) and (ii) oxidation of 4-amino-2-methoxy-5-ethyl thio benzoic acid (IX) or 4-amino-2-methoxy-5-ethyl thio methyl benzoate (X) with oxidizing agent in the presence of sodium tungstate or ammonium molybdate to give 2-methoxy-4-amino-5-ethyl-sulfonyl benzoic acid (IV) or 2-methoxy-4-amino-5-ethyl-sulfonyl methyl benzoate (XI) respectively.

### FIELD OF THE INVENTION

The present invention relates to novel process for the preparation of amisulpride.

### BACKGROUND OF THE INVENTION

Amisulpride (I), chemically known as 4-amino-N-[(1-ethylpyrrolidin-2-yl) methyl]-5-ethylsulfonyl-2-methoxy-benzamide, is an atypical antipsychotic used to treat psychosis in schizophrenia and episodes of mania in bipolar disorder. Amisulpride is a substituted benzamide. This belongs to the group of medicines known as antipsychotics. In small doses it is also used to treat depression. Amisulpride is effective in helping symptoms such as hearing voices, loss of energy, thought disturbances, difficulties communicating with others, worry, depression, overcoming feelings of wanting to be alone as well as other symptoms of schizophrenia.

Amisulpride is represented by the formula (I) as given below.

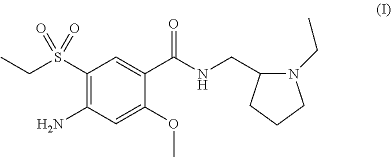 [SMILES: CCN1CCCC1CNC(=O)c1cc(S(=O)(=O)CC)c(N)cc1OC]

The product patent U.S. Pat. No. 4,401,822 describes preparation of amisulpride as shown in scheme (I)

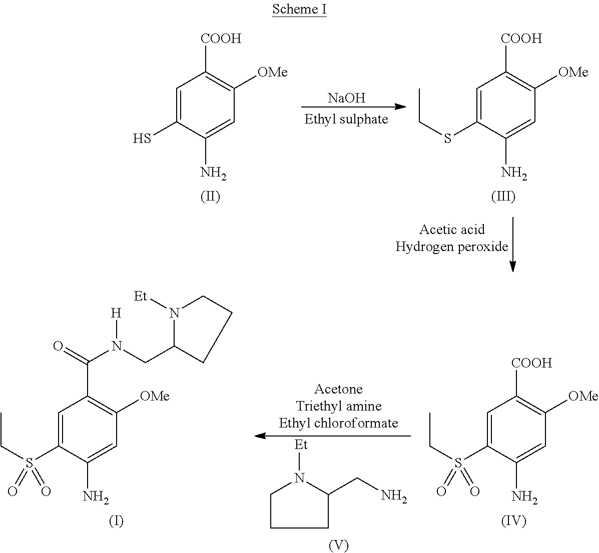

The synthesis of amisulpride involves oxidation of 2-methoxy-4-amino-5-ethyl-thio benzoic acid (III) using acetic acid and hydrogen peroxide at 40-45° C. for few hours to obtain 2-methoxy-4-amino-5-ethyl-sulfonyl benzoic acid (IV). In our attempt to repeat this reaction, we found that almost 22 hours were required for completion and the purity of compound (IV) was 87.6%.

Thus, the product patent method suffers from the disadvantages such as high reaction time, low yield and low purity.

Liu Lie et al, Jingxi Huagong Zhongjianti 2008, 38 (3), 29-32 describes the process for the preparation of 2-methoxy-4-amino-5-ethyl-sulfonyl benzoic acid (IV) as shown in scheme (II).

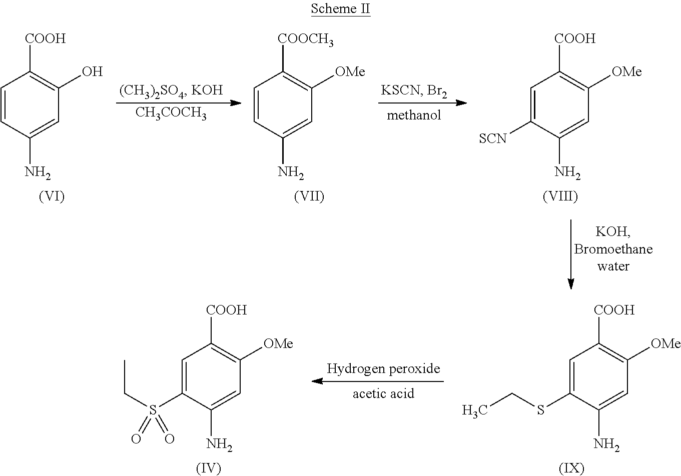

4-amino salicylic acid (VI) is treated with dimethyl sulphate in the presence of potassium hydroxide and acetone to give 4-amino-2-methoxy-methyl benzoate in 4 hours, which is further treated with potassium thiocynate to give compound of formula (VIII). 4-Amino-2-,methoxy-5-thiocyanatobenzoate (VIII) is treated with bromoethane to give 4-amino-5-ethylthio-2-methoxy benzoic acid (IX) which is further converted to 2-methoxy-4-amino-5-ethyl-sulfonyl benzoic acid (IV) via oxidation with hydrogen peroxide and acetic acid.

The yield of conversion of compound (VIII) to compound (IX) is 57% and the overall yield of compound (IV) from compound (VI) is 24% only. Thus, the above process suffers from the disadvantages such as low yield and in that it uses bromoethane which is skin and eye irritant and has carcinogenic effects.

Therefore, there is, an unfulfilled need to provide industrially feasible process for the preparation of 2-methoxy-4-amino-5-ethyl-sulfonyl benzoic acid (IV) and amisulpride (I) with higher purity and yield, since it is one of the key intermediates in the manufacture of amisulpride.

### SUMMARY OF THE INVENTION

The present invention is related to a novel process for the preparation of amisulpride (I) that involves: (i) methylation of 4-amino-salicylic-acid (VI) with dimethyl sulphate and base, optionally in presence of TBAB to obtain 4-amino-2-methoxy methyl benzoate (VII) and (ii) oxidation of 4-amino-2-methoxy-5-ethyl thio benzoic acid (IX) or 4-amino-2-methoxy-5-ethyl thio methyl benzoate (X) with oxidizing agent in the presence of sodium tungstate or ammonium molybdate to give 2-methoxy-4-amino-5-ethyl-sulfonyl benzoic acid (IV) or 2-methoxy-4-amino-5-ethyl-sulfonyl methyl benzoate (XI) respectively.

### DESCRIPTION OF THE DRAWINGS

FIG. 1: X-ray powder diffractogram (XRPD) for amisulpride obtained by the process of the present invention.

FIG. 2: Infra Red spectrum for amisulpride obtained by the process of the present invention.

### DETAILED DESCRIPTION OF THE INVENTION

The present invention provides a novel process for the preparation of amisulpride (I), that comprises the following steps:

1) methylation of 4-amino-salicylic-acid (VI) to 4-amino-2-methoxy methyl benzoate (VII), optionally in the presence of phase transfer catalyst,

2) conversion of 4-amino-2-methoxy methyl benzoate (VII) to 4-amino-2-methoxy-5-thiocyano methyl benzoate (VIII),

3) ethylation of 4-amino-2-methoxy-5-thiocyano methyl benzoate (VIII) to form 4-amino-2-methoxy-5-ethyl thio methyl benzoate (X), the intermediate compound (X) is converted to amisulpiride (I) by two routes (a) or (b):
Route (a):

4a) hydrolysis of 4-amino-2-methoxy-5-ethyl thio methyl benzoate (X) to 4-amino-2-methoxy-5-ethyl thio benzoic acid (IX),

4b) oxidation of 4-amino-2-methoxy-5-ethyl thio benzoic acid (IX) with a suitable oxidizing agent in presence of sodium tungstate or ammonium molybdate to give 2-methoxy-4-amino-5-ethyl-sulfonyl benzoic acid (IV),
Route (b):

5a) oxidation of 4-amino-2-methoxy-5-ethyl thio methyl benzoate (X) to 2-methoxy-4-amino-5-ethyl-sulfonyl methyl benzoate (XI) with a suitable oxidizing agent,

5b) hydrolysis of 2-methoxy-4-amino-5-ethyl-sulfonyl methyl benzoate (XI) to give 2-methoxy-4-amino-5-ethyl-sulfonyl benzoic acid (IV and;

6) coupling of 2-methoxy-4-amino-5-ethyl-sulfonyl benzoic acid (IV) with 1-ethyl-2-amino methyl pyrrolidine (V)

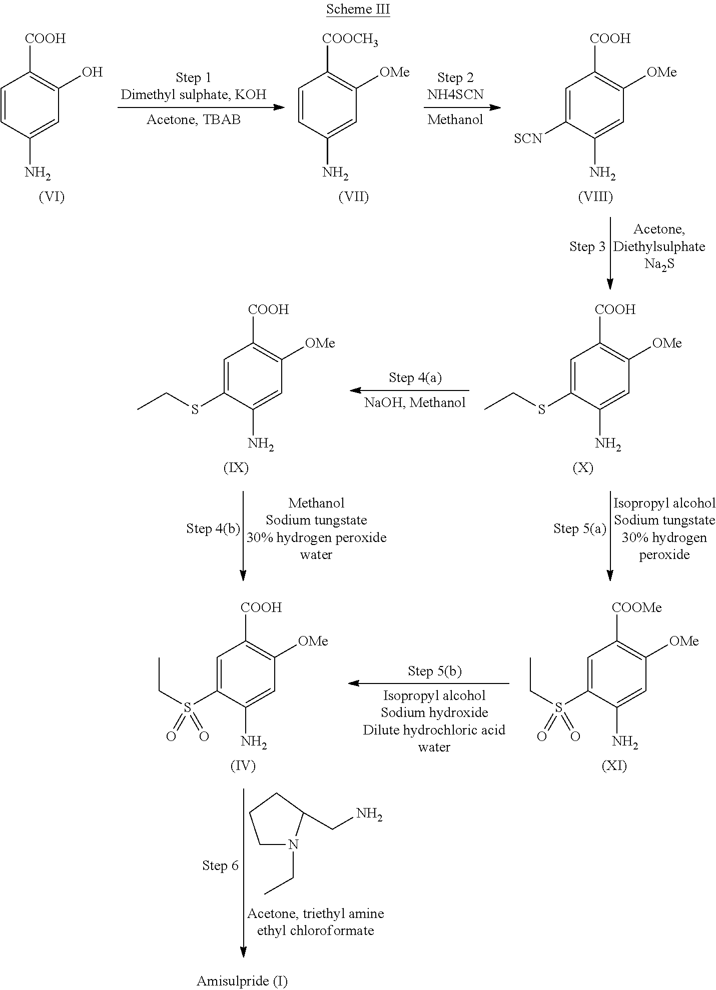

In one embodiment of the present invention, the compound 4-amino-salicylic acid (VI) is treated with dimethyl sulphate in the presence of inorganic base and suitable solvent to give 4-amino-2-methoxy methyl benzoate (VII) (step 1). The base is selected from a group of inorganic bases such as hydroxides like sodium hydroxide, potassium hydroxide, carbonates like sodium carbonate, potassium carbonate, bicarbonates like sodium bicarbonate, potassium bicarbonate etc. The preferred base being potassium hydroxide.

The reaction of step 1, can be optionally carried in the presence of a phase transfer catalyst such as tetra butyl ammonium bromide (TBAB), wherein reaction is completed in less than two hours.

The compound, 4-amino-2-methoxy methyl benzoate (VII) is converted to 4-amino-2-methoxy-5-thiocyano methyl benzoate (VIII) with of ammonium thiocyanate and bromine in the presence of methanol (step 2).

In another embodiment of the present invention, the compound 3, 4-amino-2-methoxy-5-thiocyano methyl benzoate (VIII) is subjected to ethylation with diethyl sulphate in the presence of sodium sulphide to give 4-amino-2-methoxy-5-ethyl thio methyl benzoate (X) (step 3).

The reaction of step 1 and 3 is carried out in a suitable solvent selected from the group comprising of water, alcohols like methanol, ethanol, isopropanol, esters like ethyl acetate, tertiary butyl acetate, ketones like acetone, hydrocarbons like toluene, ethers like ethyl ether, methyl ether, dioxane, tetrahydrofuran etc or mixtures thereof. The most preferred solvent is acetone.

The intermediate compound (X) is converted to amisulpride (I) by two routes : route (a) or route (b):
Route (a):

Hydrolysis of 4-amino-2-methoxy-5-ethyl thio methyl benzoate (X) to obtain 4-amino-2-methoxy-5-ethyl thio benzoic acid (IX) (step 4a) followed by oxidation of 4-amino-2-methoxy-5-ethyl thio benzoic acid (IX) with a suitable oxidizing agent in presence of sodium tungstate or ammonium molybdate to give 2-methoxy-4-amino-5-ethyl-sulfonyl benzoic acid (IV) (Step 4b).
Route (b):

Oxidation of 4-amino-2-methoxy-5-ethyl thio methyl benzoate (X) to obtain 2-methoxy-4-amino-5-ethyl-sulfonyl methyl benzoate (XI) (Step 5a) followed by hydrolysis of 2-methoxy-4-amino-5-ethyl-sulfonyl-methyl benzoate (XI) to give 2-methoxy-4-amino-5-ethyl-sulfonyl benzoic acid (IV) (Step 5b).

The compound 4-amino-2-methoxy-5-ethyl thio methyl benzoate (X) and compound 2-methoxy-4-amino-5-ethyl-sulfonyl methyl benzoate (XI) are subjected to alkaline hydrolysis to give 4-amino-2-methoxy-5-ethyl thio benzoic acid (IX) and 2-methoxy-4-amino-5-ethyl-sulfonyl benzoic acid (IV) respectively (step 4a and step 5b). The hydrolysis is carried out in the presence of base selected from a group of inorganic bases such as hydroxides like sodium hydroxide, potassium hydroxide, carbonates like sodium carbonate, potassium carbonate, bicarbonates like sodium bicarbonate potassium bicarbonate etc. The preferred base being sodium hydroxide.

The solvent for hydrolysis is selected from water, methanol, ethanol, isopropanol, dioxane, tetrahydrofuran, dimethylformamide, dimethylsulfoxide, acetonitrile, acetone, methyl ethyl ketone etc or mixtures thereof The most preferred solvent for hydrolysis is methanol or isopropanol.

In yet another preferred embodiment of the present invention is the oxidation of 4-amino-2-methoxy-5-ethyl thio benzoic acid (IX) and 4-amino-2-methoxy-5-ethyl thio methyl benzoate (X) in the presence of sodium tungstate or ammonium molybdate to give 2-methoxy-4-amino-5-ethyl-sulfonyl benzoic acid (IV) or 2-methoxy-4-amino-5-ethyl-sulfonyl methyl benzoate (XI) respectively with a suitable oxidizing agent. The oxidizing agent is selected from hydrogen peroxide, perbenzoic acid, meta chloro perbenzoic acid, per acetic acid, sodium hypochlorite, sodium per borate tetrahydrate etc. The preferred oxidizing agent is hydrogen peroxide.

The oxidizing agent was used in the range of 0.1-5% by weight, preferably 0.5 to 3% by weight.

The oxidation reaction is carried out in a suitable solvent selected from the group comprising of water, alcohols like methanol, ethanol, isopropanol, esters like ethyl acetate, tertiary butyl acetate, hydrocarbons like toluene, ethers like ethyl ether, methyl ether, dioxane, tetrahydrofuran, dimethylformamide, dimethylsulfoxide, acetonitrile, acetone, methyl ethyl ketone etc or mixtures thereof. The most preferred solvent for oxidation is methanol or isopropanol.

The oxidation reaction is typically carried out for 1-4 hours at −10 to 100° C., preferably at 20-50° C.

Coupling of the 2-methoxy-4-amino-5-ethyl-sulfonyl benzoic acid (IV) with 1-ethyl-2-amino methyl pyrrolidine (V) is carried out with triethyl amine and ethyl chloroformate in acetone as solvent to give amisulpride.

The amisulpride obtained is optionally purified by crystallization from acetone.

The purity of amisulpride obtained after crystallization is ≧99% and the yield is in the range of 75-80%.

The aforementioned process for the preparation of amisulpride (I) has the following several

advantages over prior art methods:

purity of amisulpride is ≧99%,

yield of amisulpride is more,

avoids use of irritants like bromo ethane,

simple and quick process,

easy to scale up and

economical process,

The principles, preferred embodiments, and modes of operation of the present invention have been described in the foregoing examples. The invention, which is intended to be protected herein, however, is not to be construed limited to the particular forms disclosed, since these are to be regarded as illustrative rather than restrictive. Variations and changes may be made by those skilled in the art, without departing from the spirit of the invention.

### Examples

### Example 1

Preparation of 4-amino-2-methoxy methyl benzoate (VII)

4-Amino salicylic acid (VI) (2 kg) was added in acetone (12 lit) under stirring. Tetrabutyl ammonium bromide (2.09 kg) was added followed by addition of potassium hydroxide (2.18 kg) and the reaction mass was stirred. To the reaction mass dimethyl sulphate (3.89 kg) was added dropwise at 25-35° C. Stirring was continued at 25-35° C. for 60 min. Reaction mass was quenched in prechilled water (30 Lit) at 0-5° C. Reaction mass was stirred and solid obtained by filtration under suction. Solid was washed with water and dried under suction. The wet solid was leached with methanol (2 Lit) at 60-65° C. The reaction mass was cooled to 0-5° C. and solid was obtained by filtration, dried under vacuum.

Yield : 72%

Purity: 98%

### Example 2

Preparation of 4-amino-2-methoxy-5-thiocyano methyl benzoate (VIII)

4-Amino-2-methoxy-methyl benzoate (VII) (1.5 Kg) was added in methanol (7.5 lit.) under stirring followed by addition of ammonium thiocyanate (1.49 Kg). Reaction mass was cooled to 5-10° C. Bromine (1.97 kg, 12.43 mol) diluted with methanol (7.5 Lit.) was slowly added by maintaining the temperature below 10° C. The temperature was raised to 15° C. along with stirring for 3-4 hrs. The reaction mass was then cooled to 0-5° C. Solid was filtered and washed with excess of water.

Yield : 82%

Purity: 90%

### Example 3

Preparation of 4-amino-2-methoxy-5-ethyl thio methyl benzoate (X)

4-Amino-2-methoxy-5-thiocyano methyl benzoate (VIII) (1.62 Kg) was added in acetone (7.5 lit.) and water (7.5 Lit.) under stirring. Na₂S (1.26 Kg) in water (7.5 Lit.) solution was added in reaction mass at 5-10° C. followed by addition of diethyl sulphate (1.19 Kg, 7.75 mol) slowly at 5-10° C. The reaction mass was stirred at 10-15° C. for 2-3 hrs. The solid was filtered and recrystalised from methanol to get pure 4-amino-2-methoxy-5-ethylthio methyl benzoate.

Yield : 74%

Purity: 99%

### Example 4

Preparation of 4-amino-2-methoxy-5-ethyl thio methyl benzoic acid (IX)

Methanol (4.0 L) was added to 4-amino-2-methoxy-5-ethyl thio methyl benzoate (1.0 Kg) (X) under stirring. Sodium hydroxide solution in water (0.82 Kg+0.82 lit) was added to the mixture and the reaction was heated to 65° C. The reaction mass was stirred and the pH of the reaction mass was adjusted till pH=4.0−4.5 by using diluted HCl (1:10) at 5-10° C. The solid was filtered and dried under vacuum.

Yield : 96%

Purity: 99%

### Example 5

Preparation of 4-amino-2-methoxy-5-ethyl sulphonyl benzoic acid (IX)

To a solution of sodium tungstate (7.26 g) in water (1.0 L) hydrogen peroxide was added (1.25L). Reaction mass was stirred and cooled to 10° C. In another flask, 4-amino-2-methoxy-5-ethyl thio methyl benzoic acid (IX) (1.0 Kg) was dissolved in methanol (4.0 L) at 40-45° C. and the solution was added in above reaction mass at 10-15° C. After completion of reaction, reaction mass was poured in prechilled sodium thiosulphate solution. Reaction mass was cooled, stirred and filtered.

Yield : 74%

Purity: 99%

### Example 6

Preparation of 2-methoxy-4-amino-5-ethyl-sulfonyl benzoic acid (IV)

30% Hydrogen peroxide was slowly added to a solution of the 4-amino-2-methoxy-5-ethylthio methyl benzoate (X) (1.21 Kg) in isopropyl alcohol (4.84 Lit.) containing sodium tungstate (0.0082 Kg) as a catalytic amount at ambient temperature. The mixture was stirred at 40-45° C. for 3-4 hrs then cooled to 5-10° C. 5% sodium thiosulphate solution (0.06 Kg in 18.15 lit. water) was added to reaction mixture. Reaction mass was stirred for 60 min and 2-methoxy-4-amino-5-ethyl-sulfonyl methyl benzoate (XI) was obtained in situ. To the reaction mixture was added sodium hydroxide (1.00 Kg, in 10 lit. water). The temperature was raised up to 60-65° C. and stirred the reaction mixture for 2-3 hrs. The reaction mass was cooled and adjusted pH 4.0-4.5 by using diluted hydrochloric acid (1:10). The product was isolated by filtration under suction.

Yield : 82% p Purity: 99%

### Example 7

Preparation of 2-methoxy-4-amino-5-ethyl-sulfonyl benzoic acid (IV)

2-Methoxy-4-amino-5-ethyl thio benzoic acid (100 g) was dissolved in acetic acid 440 ml) at 55-60° C. The reaction mixture was cooled to 35° C. Hydrogen peroxide (150 ml) was added slowly. The reaction mixture was slowly heated to 80° C. and cooled to 40° C. The reaction was stirred till completion or about 20 hours. The reaction mass was cooled to 10° C. and filtered. The solid collected was dissolved in water (500 ml) and ammonia solution (80 ml) and precipitated using concentrated hydrochloric acid (40 ml). The reaction mixture was cooled, stirred, filtered and dried.

Yield : 75%

Purity: 97.83%

### Example 8

Preparation of 2-methoxy-4-amino-5-ethyl-sulfonyl benzoic acid (IV)

2-Methoxy-4-amino-5-ethyl thio benzoic acid (100 g) was added in acetic acid (440 ml) followed by addition of per acetic acid (334.8 g) at room temperature. The reaction mass was heated to 40-45° C. and stirred for about 5 hours. The reaction mass was cooled and filtered. Isolated solid was dissolved in water (500 ml) and ammonia solution (80 ml) and precipitated using concentrated hydrochloric acid (40 ml). The reaction mass was cooled to 0-5 ° C., stirred, filtered and dried.

Yield: 30%

Purity: 98.51%

### Example 9

Preparation of 2-methoxy-4-amino-5-ethyl-sulfonyl benzoic acid (IV)

Ammonium molybdate (27.2 g) was dissolved in water (1 liter) and hydrogen peroxide (1.5 liter) was added to the mixture. In another flask 2-methoxy-4-amino-5-ethyl thio benzoic acid (1 kg) was dissolved in hot methanol (4 liters). The solution was cooled to room temperature and slowly added to the oxidizing mixture below 25-30° C. The reaction mass was stirred for about 4 hours. The reaction mass was poured in pre cooled sodium thiosulphate solution. The mixture was cooled, stirred, solid was filtered and dried.

Yield: 75%

Purity: 99.44%

### Example 10

Preparation of 2-methoxy-4-amino-5-ethyl-sulfonyl benzoic acid (IV)

Acetic acid (400 ml) was added to 2-methoxy-4-amino 5 ethyl thio benzoic acid (100 g) at room temperature. The reaction mass was stirred to obtain a slurry. Sodium per borate tetrahydrate (142.33 g) was added to the mixture. The reaction mass was heated to 40-45° C. and was stirred, filtered and dried.

Yield: 60%

### Example 11

Preparation of 2-methoxy-4-amino-5-ethyl-sulfonyl benzoic acid (IV)

Sodium tungstate (0.726 g) was dissolved in water (100 ml) and hydrogen peroxide 30% (250 ml) was added to the mixture. In another flask 2-methoxy-4-amino 5 ethyl thio benzoic acid (100 g) was dissolved in hot methanol (400 ml) at 40-45° C. The solution was cooled to room temperature and slowly added to the above oxidizing mixture below 25-30° C. The reaction mass was stirred for about 5 hours. The reaction mass was poured in pre cooled sodium thiosulphate solution at 5-15° C. The mixture was cooled, stirred, solid was filtered and dried.

Yield: 80%

### Example 12

Preparation of 2-methoxy-4-amino-5-ethyl-sulfonyl benzoic acid (IV)

Water (500 ml) was added to 2-methoxy-4-amino 5 ethyl thio benzoic acid (100 g) and mixture was dissolved. Ammonium molybdate (2.72 g) was added to the reaction mixture. The mixture was cooled to 8-10° C. 30% hydrogen peroxide (250 ml) was added slowly at same temperature. The reaction was further stirred at room temperature. The reaction mass was cooled to 0-5° C. and stirred. The reaction mass was filtered and washed with water. The solid was suck dried.

Yield: 68%

### Example 13

Preparation of crude amisulpride

To a stirring mixture of 4-amino-2-methoxy-5-ethyl sulphonyl benzoic acid (IV) and acetone (5.0 L) at 0-5° C., triethyl amine (0.405 Kg) was added and stirred followed by addition of ethyl chloroformate (0.368 Kg). N-ethyl-2-amino methyl pyrrolidine (0.627 Kg) was added to the reaction mass at 5-10° C. Temperature of reaction mass was raised to 25-30° C. and stirred for 120 min. To the same reaction mass triethyl amine (0.405 Kg) and ethyl chloroformate (0.368 Kg) was added with maintaining the temperature. Reaction mass was stirred for 120 min. After completion of reaction, water (4.0 L) was added. Reaction mass was filtered and washed with water (2.0 L). Filtrate was collected and water was added (9.0 L). pH of the reaction mass was adjusted to 10.8-11.2 by using 20% NaOH solution. Reaction mass was stirred for 240-300 min, filtered and washed with water. Solid was dried under vacuum

Yield : 70%

Purity: 98%

### Example 14

Purification of amisulpride

Amisulpride (1 kg) was charged in acetone (6 liters) and the reaction mixture was heated till a clear solution was obtained. Slurry of activated carbon (0.1 kg in 1 liter) was added in acetone. The reaction mass was stirred at 50-55 ° C. for 60 minutes and filtered hot. The filtrate was concentrated and further heated to dissolve the solid. The reaction mass was cooled to 0-5° C., stirred and filtered. The precipitated solid was washed with acetone and dried.

Yield: 750 gm (75%)

HPLC purity: 99.8% (quantitative)

M.P.: 125° C.

DSC: shows endotherm at 133° C.

Particle size: d₁₀=0.637, d₅₀=6.0, d₉₀=13.325 microns

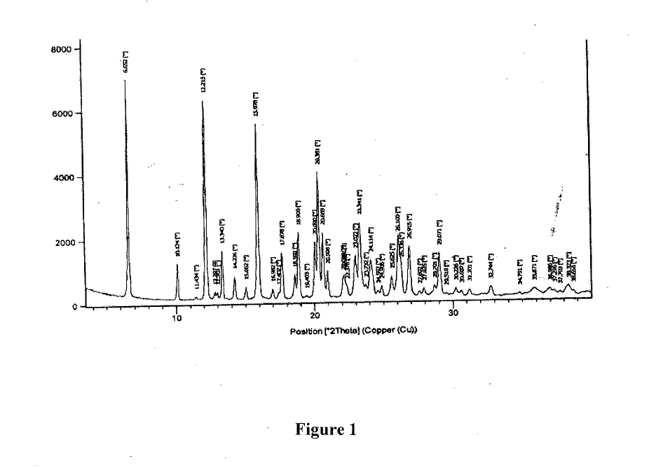

PXRD is shown in FIG. 1.

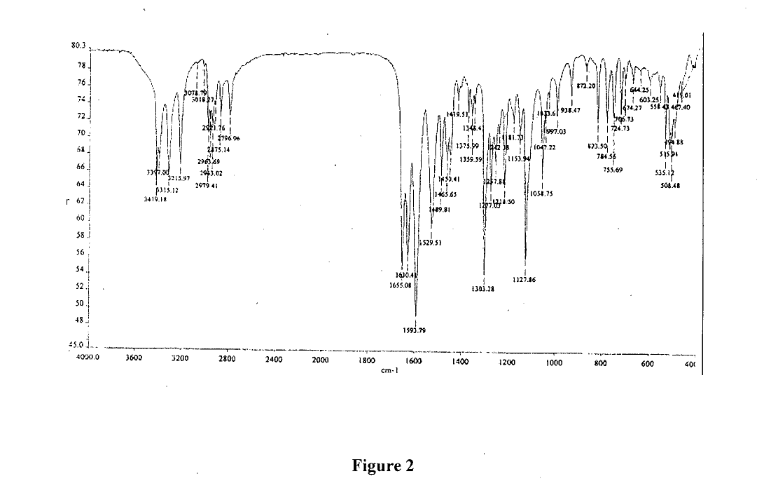

IR is shown in FIG. 2.

1) A process for the preparation of amisulpride (I) which involves following steps:
1) methylation of 4-amino-salicylic-acid (VI) to 4-amino-2-methoxy methyl benzoate (VII), optionally in the presence of phase transfer catalyst,
2) conversion of 4-amino-2-methoxy methyl benzoate (VII) to 4-amino-2-methoxy-5-thiocyano methyl benzoate (VIII),
3) ethylation of 4-amino-2-methoxy-5-thiocyano methyl benzoate (VIII) to form 4-amino-2-methoxy-5-ethyl thio methyl benzoate (X),
the intermediate compound (X) is converted to amisulpiride (I) by two routes (a) or (b):
Route (a):
4a) hydrolysis of 4-amino-2-methoxy-5-ethyl thio methyl benzoate (X) to 4-amino-2-methoxy-5-ethyl thio benzoic acid (IX),
4b) oxidation of 4-amino-2-methoxy-5-ethyl thio benzoic acid (IX) with a suitable oxidizing agent in presence of sodium tungstate or ammonium molybdate to give 2-methoxy-4-amino-5-ethyl-sulfonyl benzoic acid (IV),
Route (b):
5a) oxidation of 4-amino-2-methoxy-5-ethyl thio methyl benzoate (X) to 2-methoxy-4-amino-5-ethyl-sulfonyl methyl benzoate (XI) with a suitable oxidizing agent,
5b) hydrolysis of 2-methoxy-4-amino-5-ethyl-sulfonyl methyl benzoate (XI) to give 2-methoxy-4-amino-5-ethyl-sulfonyl benzoic acid,(IV and;
6) coupling of 2-methoxy-4-amino-5-ethyl-sulfonyl benzoic acid (IV) with 1-ethyl-2-amino methyl pyrrolidine (V)

2) A process according to claim 1 wherein step 1 is carried out in the presence of dimethyl sulfate and potassium hydroxide.

3) A process according to claim 1 wherein, the phase transfer catalyst used in step 1 is tetrabutyl ammonium bromide.

4) A process according to claim 1 wherein, step 2 is carried out in the presence of ammonium thiocyanate and methanol.

5) A process according to claim 1 wherein, step 3 is carried in the presence of diethyl sulfate and sodium sulphide.

6) The process according to claims 1-5 wherein, the oxidizing agent used in step 4b and step 5a is selected from hydrogen peroxide, perbenzoic acid, meta chloro perbenzoic acid, per acetic acid, sodium hypochlorite, sodium per borate tetrahydrate.

7) The process according to claim 6 wherein, the most preferred oxidizing agent is hydrogen peroxide.

8) The process according to claim 1-6 wherein the solvent used for oxidation in step 4b and step 5a is selected from water, alcohols like methanol, ethanol, isopropanol, esters like ethyl acetate, tertiary butyl acetate, hydrocarbons like toluene, ethers like ethyl ether, methyl ether, dioxane, tetrahydrofuran, dimethylformamide, dimethylsulfoxide, acetonitrile, acetone, methyl ethyl ketone or mixtures thereof.

9) The process according to claim 8 wherein, the most preferred solvent is methanol or isopropanol.

10) A process for oxidation of 4- amino-2-methoxy-5-ethyl thio benzoic acid (IX) in the presence of sodium tungstate or ammonium molybdate.

11 ) A process for oxidation of 4-amino-2-methoxy-5-ethyl thio methyl benzoate (X) in the presence of sodium tungstate or ammonium molybdate.

12) A process of claims 10 and 11 wherein, the oxidizing agent is selected from hydrogen peroxide, perbenzoic acid, meta chloro perbenzoic acid, per acetic acid, sodium hypochlorite, sodium per borate tetrahydrate.

13) The process according to claim 12 wherein, the most preferred oxidizing agent is hydrogen peroxide.

14) The process according to claims 10 and 11 wherein the solvent used for is selected from water, alcohols like methanol, ethanol, isopropanol, esters like ethyl acetate, tertiary butyl acetate, hydrocarbons like toluene, ethers like ethyl ether, methyl ether, dioxane, tetrahydrofuran, dimethylformamide, dimethylsulfoxide, acetonitrile, acetone, methyl ethyl ketone or mixtures thereof.

15) The process according to claim 14 wherein, the most preferred solvent is methanol or isopropanol.

16) The process for methylation of 4-amino-salicylic-acid (VI) to 4-amino-2-methoxy methyl benzoate (VII) with dimethyl sulfate and potassium hydroxide in presence of tetrabutyl ammonium bromide.

17) The process for the preparation of amisulpride as described by the foregoing examples.
